(19) United States
(12) Patent Application Publication
(10) Pub. No.: US 2020/0222307 A1
(43) Pub. Date: Jul. 16, 2020

(54) DELIVERY OF CANNABIS COMPOSITION

(71) Applicants: Shaiw-Wan Liu, Honeybrook, PA (US); Phyllis Hsieh, Honeybrook, PA (US)

(72) Inventors: Shaiw-Wan Liu, Honeybrook, PA (US); Phyllis Hsieh, Honeybrook, PA (US)

(21) Appl. No.: 16/741,615

(22) Filed: Jan. 13, 2020

Related U.S. Application Data
(60) Provisional application No. 62/791,130, filed on Jan. 11, 2019.

(51) Int. Cl. A61K 8/9789; A61K 8/34; A61K 8/92; A61K 8/37; A61K 8/73; A61Q 19/00; A61K 31/05

(52) U.S. Cl. CPC A61K 8/9789; A61K 8/345; A61K 8/922; A61K 31/05; A61K 8/73; A61K 8/927; A61Q 19/00; A61K 8/37

(57) ABSTRACT

A composition comprising a Hsieh Enhancer (U.S. Pat. No. 5,023,252) and (A) at least one compound which has psycoactive properties and the source of which is cannabis; or (B) at least one compound which approves the appearance of the skin of an individual.

### CROSS REFERENCE TO RELATED APPLICATION

This application claims the benefit of U.S. Provisional Application No. 62/791,130, filed Jan. 11, 2019 which application is incorporated herein by reference.

The present invention relates to a composition comprising a material which has psycoactive properties and a compound which enhances the rate of passage of the material into the body of an individual (referred to hereafter for convenience as “enhancer”). More particularly, the present invention relates to a composition which comprises an enhancer and a psycoactive chemical substance that has an effect on the function of the brain by acting on the central nervous system of the individual.

The present invention is described in connection with the use of the enhancer to deliver into the body materials which are typically derived from plants, for example, cannabis.

The present invention is also related to the use of a composition which contains an enhancer and a “cannabis” compound for the purpose of improving the quality of skin by applying the composition to the surface of skin.

Cannabis is a generic term that refers to a flowering plant, for example, marijuana and hemp, that is characterized by having a stiff upright stem bearing divided serrated leaves and glandular hairs. It is a generic term that encompasses many species of compounds, including compounds that have psychoactive properties and other properties. The term “cannabis” is used herein to encompass within its meaning various species of compounds that have psychoactive properties and skin-improving properties of which it is a source.

### SUMMARY OF THE INVENTION

The present invention provides a composition comprising a Hsieh Enhancer (U.S. Pat. No. 5,023,252) and at least one compound which has psycoactive properties or skin-improving properties and the source of which is cannabis. The term “Cannabis” is used herein to refer to two plants, namely marijuana and hemp, each plant containing one or more chemical compounds that have psycoactive or skin-improving properties.

The cannabis plant is a source of marajuana which, for example, can produce a sense of euphoria or an impairment of judgment. The cannabis plant is a source also of hemp from which drugs and a variety of industrial products can be made, for example, commercially available fiber, paper, textiles, and building materials.

### DETAILED DESCRIPTION OF INVENTION RELATING TO PSYCOACTIVE DEVELOPMENT

Speaking generally, psychoactive compounds are recognized to have a significant effect on the mental processes of an individual, for example, producing in the individual a sense of euphoria and impairment of judgment; use can affect how the brain and nervous systems work. Although the use of psychoactive compounds can affect adversely the behavior of an individual, it has been recognized that their use can be also helpful for treating certain medical conditions and for recreational purposes. Accordingly, the present invention can be used to advantage by the provision of cannabis-containing compositions that include an enhancer.

A preferred cannabis for use in the practice of the present invention is cannabidiol (hereafter “CBD”) a source of which is hemp that is itself a species of cannabis. CBD is recognized by certain authorities to have psychoactive properties that are considered to be of value in treating individuals; however, problems are encountered in delivering CBD to the body in effective quantities. Accordingly, combining CBD with the enhancer hereof is advantageous.

The amount of cannabis comprising the present composition will depend on many factors, including, for example, the particular species of cannabis used and the desired effect to be achieved on the individual who is to be treated, the particular enhancer and amount used, and the particular form of the composition applied to the body of the individual. It is believed that an amount of cannabis within the range of about 0.01 wt. % to about 15 wt. % will be effective for most applications.

The preferred enhancer for use in the composition of the present invention is a member of the class of compounds which have become known in the art as “Hsieh enhancers”, for example, as described in U.S. Pat. No. 5,023,252 to Hsieh. This patent describes that Hsieh enhancers function as transdermal- or transmembrane-delivering compounds that enhance the delivery of a drug from the surface of the skin or nasal membrane into the blood stream of the individual. Accordingly, in accordance with the disclosure of the Hsieh patent, the Hsieh enhancers are used in applications in which they are combined with a drug, which to be effective, must be delivered systematically into the blood stream of the recipient.

Examples of Hsieh enhancers that can be used in the present composition are 3-methylcyclopentadecanone (muscone), 9-cycloheptadecen-1-one (civetone), cyclohexadecanone, and cyclopentadecanone (normuscone). The use of pentadecalactone is preferred.

As described below, the composition of the present invention can be applied to the body in various forms, for example, in the form of solids dispersed in a liquid carrier or dissolved liquid ingredients dispersed in a liquid carrier (for example, various types of emulsions). For this purpose, the enhancer can be dissolved, dispersed, suspended or solubilized in suitable solvents, such as alcohols, oils, glycerol, ethylene glycol propylene glycol, hexane, acetone, Freon, water or mixtures thereof.

The amount of enhancers comprising the present composition will depend on many factors, including, for example, the particular species of cannabis used and the amount thereof, the desired effect to be achieved on the individual who is to be treated, and the particular form of the composition applied to the body of the individual. It is believed that an amount of enhancer within the range of about 0.1 wt. % to about 20 wt. % will be effective for most applications.

### Detailed Description of Invention Relating to Skin Improvement

The present development includes also the use of the Hsieh Enhancer to improve the qualities of skin, for example, smoother appearance of the skin, reduce the development of skin inflammation, for example, acne and other types of inflammation. Reference is made to U.S. Pat. No. 5,731,303, particularly the paragraph bridging columns 20 and 21 and continuing to column 28.

Compounds which can be used in treatment of the skin include psycoactive compounds, for example, those present in marijuana. A preferred skin-treating compound is cannabidol (CBD) a source of which is hemp.

The amount of cannabis contained in the skin-enhancing composition will depend on many factors, including, for example, the particular species of cannabis used, the particular form of the composition used by the individual and the effects which are desired. It is believed that an amount of cannabis within the range of about 0.01 wt. % to about 15 wt. % will be effective for most applications.

The enhancer for use in the skin-treating composition is a member of the class of “enhancing” compounds which are described in the aforementioned U.S. patents.

Examples of Hsieh enhancers that can be used in the present composition are 3-methylcyclopentadecanone (muscone), 9-cycloheptadecen-1-one (civetone), cyclohexadecanone, and cyclopentadecanone (normuscone). The use of pentadecalactone is preferred and is referred to hereafter as “CPE-215”.

The amount of enhancer or mixture of enhancers comprising the skin-treating composition will depend on many factors, including, for example, the particular species of cannabis used and the amount thereof, the conditions to be treated on the individual, and the particular form of the composition applied to the skin of the individual, for example, other ingredients and amounts thereof which are included in the composition to formate a composition of the desired form, as described below.

As stated above, the cannabis/enhancer compositions of the present invention can be applied to the body in various forms. Examples below are illustrative of various forms of composition which contain other ingredients. The compositions include lotions, creams, and gels, and ingredients which include surfactants, emulsifiers, preservatives, solvents, and thickeners. Amounts of ingredients are wt. % based on the total weight of the composition.

Attention is directed to the publication which is referred to below. It contains a wealth of information on compounds which are present in marijuana and hemp and test evaluations of properties of such compounds: HTTP://CALGARYCMMC.COM/CHEMICAL_COMPOSITION.HTM.

### Example 1

Hemp Cream

| | | |
|---|---|---|
| deionized water | Q.S. to 100% | |
| cetyl esters | 1%-8% | emulsifier |
| glycerine | 1%-10% | solvent |
| vegetable oil | 1%-30% | Carrier base |
| CPE-215 | 0.1%-10% | enhancer |
| hemp oil | 0.1%-20% | cycoactive |
| anthan gum | 1%-6% | thickener |
| phenoxyethanol, methlparaben, | 1%-2% | preservatives |
| butylparaben, | | |

### Example 2

Hemp Lotion

| | | |
|---|---|---|
| deionized water | Q.S. to 100% | |
| cetyl alcohol | 1%-10% | emulsifier |
| glycerine | 1%-10% | solvent |
| vegetable oil | 1%-30% | carrier base |
| CPE-215 | 0.1%-10% | enhancer |
| hemp oil | 10%-5% | cycoactive |
| acrylates copolymer | 1%-10% | thickener |
| phenoxyethanol, methlparaben, | 1%-2% | preservatives |
| butylparaben, | | |

### Example 3

Hemp Oily Gel

| | | |
|---|---|---|
| vegetable oil | 75%-90% | carrier base |
| polyglycerol-3 Beewax | 1%-10% | emulsifier |
| hemp oil | 0.1%-20% | cycoactive |
| CPE-215 | 0.1%-10% | enhancer |

### Example 4

Hemp Oil

| vegetable oil | Q.S to 100% | carrier base |
|---|---|---|
| hemp oil | 0.1%-20% | cycoactive |
| CPE-215 | 0.1%-10% | enhancer |

### Example 5

Hemp Lipo Serum

| vegetable oil | Q.S. to 100% |
|---|---|
| hemp oil | 0.1%-20% |
| CPE-215 | 0.1%-10% |
| Vitamin E oil | 1%-10% |

### Example 6

Hemp Serum

| deionized water | Q.S. to 100% |
|---|---|
| ethoxydiglycol | 1%-10% |
| glycerine | 1%-10% |
| tween 20 | 0.1%-5% |
| CPE-215 | 0.1%-10% |
| Xanthan gum | 0.5%-5% |
| phenoxvethanol | 1%-2% |

We claim:

1. A composition comprising a Hsieh Enhancer and (A) at least one compound which has psycoactive properties and the source of which is cannabis; or (B) at least one compound which approves the appearance of the skin of an individual.

2. A composition according to claim 1 wherein the source of the compound of (A) is marijuana.

3. A composition according to claim 1 wherein the source of the compound of (A) is hemp.

4. A composition according to claim 3 wherein the “hemp” compound is cannabidiol.
